(19) United States
(12) Patent Application Publication
(10) Pub. No.: US 2014/0324014 A1
(43) Pub. Date: Oct. 30, 2014

(54) SAFETY FLUID CONNECTOR FOR AN EXTRACORPOREAL FLUID LINE

(71) Applicant: TRANSCUTAN AB, Huddinge (SE)

(72) Inventors: Dan Lundgren, Hovas (SE); Rickard Nyman, Uppsala (SE)

(21) Appl. No.: 14/360,138

(22) PCT Filed: Nov. 30, 2012
(86) PCT No.: PCT/SE2012/000196

§ 371 Date: May 22, 2014

(30) Foreign Application Priority Data
Dec. 5, 2011 (SE) 1100891-9

(51) Int. Cl. A61M 39/10

(52) U.S. Cl. CPC A61M 39/10
USPC 604/500; 604/533

(57) ABSTRACT

A safety fluid connector for exchange of fluid with the human body of a substantially non-flexible polymer material has a first open end and a second open end connected by a fluid passage and comprises a predetermined breaking point designed to fracture unintentionally at a predetermined breaking force of from 1N to 20 N. Also disclosed is a medical tubing comprising the connector, a method of fluid exchange with the human body and the use of the tubing in the method.

### FIELD OF THE INVENTION

The invention relates to a safety device for an extracorporeal fluid line, to a fluid line comprising the device and to a corresponding method of use.

### BACKGROUND OF THE INVENTION

Extracorporeal fluid lines of various kind for providing fluid to or from a person are known in the art. An example for such fluid lines is one for connection to a transcutaneous port.

Transcutaneous ports are devices for implantation into a patient in need of repeated reliable administration of nutrients, medications, water, etc. to the gastrointestinal tract, such as disclosed in EP 1 492 589 B1. The intracorporeal connection between the port and the gastrointestinal tract is provided by an enteral catheter. The nutrient or medication for administration is a fluid state, such as an aqueous solution or suspension. It is stored in a container, from whence it is fed via a fluid line to the port by means of pump, in particular a roller pump acting on a flexible polymer tube connecting the container with the port.

The fluid line is a soft polymer tube, which has connectors at its both ends, such as, for instance, a male connector at one end and a female at its other end. The connectors can, for instance, be of Luer Lock™ type. They can be coupled with corresponding proximal and distal connectors on the port and the container, respectively, to provide fluid communication between the container and the port. The tubing of the fluid line need not consist of a single tube but may comprise two or more sections of different material and/or diameter. By the port being integrated into soft tissue of the patient the tubing becomes firmly attached to the patient, from which it cannot be easily severed except by uncoupling the proximal connector.

A problem inherent with such tubing is accidental, unintentional stress exerted on the connection between the patient and the container/pump assembly, for instance by the patient moving away from the assembly. The stress force acting on the port will pull the port away from the patient, that is, from its implanted state. The pulling force may damage the integration of the port with the adjacent tissue, causing bleeding and inflammation. A sufficiently high force of this kind may even result in the port being withdrawn from the patient, putting the health of the patient at severe risk.

The aforementioned problem is not limited to transcutaneous ports for enteral catheters but is inherent to all kind of catheters for fluid administration to or exchange with the a patient, such as catheters for intravenous or peritoneal dialysis, provided that the catheter is firmly attached to the patient. Attachment can be by implantation but also by medical tape, rubber bands, wrist cuffs, bandages, etc.

AU 657714 B2 discloses a tubing administration set for use in peritoneal dialysis. The set is designed to allow separation of a tubing after fluid delivery by breaking it intentionally at a scoring on the outer surface of the tubing by application of a bending force applied by an operator.

JP 6225990 A discloses a tube connector for use with a medical bag comprising a wall portion thinned by a circular grove designed for intentional breaking of the connector by an operator.

US 2004/0067161 A1 discloses a medical line comprising a breakable coupling device for use in peritoneal dialysis. After completion of the treatment the coupling device of the used medical line set (which is to be discarded) is intentionally separated into two pieces by an operator breaking the predetermined breaking section through twisting or bending.

### OBJECTS OF THE INVENTION

An object of the invention is provide a safety means for reducing the risk of a patient being hurt by unintended stress exerted on a tubing, such as a catheter tubing, inserted into the human body or connected to an implanted device or a transcutanous, pernasal, peroral, peraural, perurethral or peranal catheter, with the proviso that the tubing, the catheter or the device is firmly attached to the body.

In particular, an object of the invention to provide a safety means for reducing the risk of a patient being hurt by unintended stress exerted on a tubing connecting a transcutaneous port implanted in a patient with a fluid container/pump assembly.

An additional object of the invention is to provide a tubing of such kind comprising the safety means.

Another object is to provide a method of transcutaneous, peroral, peraural, pernasal, peranal or perurethral fluid exchange.

Further objects of the invention will become evident from the following summary of the invention, preferred embodiments thereof illustrated in a drawing, and the appended claims.

### SUMMARY OF THE INVENTION

According to the present invention is provided a safety means of the aforementioned kind in form of a fluid connector of a substantially non-flexible polymer material comprising a predetermined breaking or fracture point such as a failure notch and having two open ends connected by a fluid passage. In this application, “predetermined breaking point” indicates a design of the fluid connector causing the connector to reliably break at that point upon application of a predetermined breaking force acting on the two ends of the connector drawing them apart. If not otherwise indicated, in this application “connector” refers to the fluid connector of the invention. In this application “fluid exchange” comprises infusing fluid into a patient, removing fluid from a patient, and exchanging the fluid of a patient, such as in hemodialysis or peritoneal dialysis. The device of the invention differs from known tubing or tubing connectors provided with predetermined breaking points by being designed to break at accidental loads so as to prevent the patient from being put at risk by withdrawal of the transcutaneous port or catheter or other device for fluid exchange with the human body. The loads at which the device is designed to break are, by necessity, substantially lower than the loads required to break known fluid connection devices, since the latter are designed to be intentionally not accidentally broken.

In a preferred embodiment of the invention the predetermined breaking force acts on said ends by two flexible tubes connected to connector portions extending from said ends, in particular non-releaseably connected, such as by welding, gluing or friction. The connections between the flexible tubes and the respective end portions of the connector are capable of withstanding a force seeking to withdraw them from the connector that is a multiple of the breaking force, such as a tenfold or fiftyfold or even hundredfold breaking force. In another preferred embodiment of the invention the proximal end of the safety fluid connector is connected directly to an implant or a catheter for providing transcutaneous, peroral, pernasal, peraural, perurethral or peranal access to the human body, such as a venous or gastrointestinal or peritoneal catheter or a transcutaneous port.

A preferred breaking force is from 1 N to 20 N, more particularly from 5 N to 20 N, in particular from 5 N to 15 N, most particularly about 5 N. The appropriate breaking force or breaking force range will vary for applications of different kind; for a particular application can be easily experimentally determined by a person skilled in the art. An appropriate breaking force is one that prevents withdrawal of the implanted device from the human body including one that prevents its attachment to the human body being jeopardized by, for instance, weakening the integration of the implant with surrounding tissue.

According to another preferred aspect of the invention the connector comprises a V-shaped portion comprising one or more breaking notches. It is preferred for the angle between the arms of the V-shaped portion to be from 15° to 75°, in particular from 5° to 60°, in particular from 10° to 45°. It is preferred for the one or at least one of the more than one breaking notches to be radial notch or a substantially radial notch, that is a notch not deviating more than about 15° from a radial plane. The one or more notches can be provided at one or both arms. Alternatively, at least one notch is provided at the joining section of the arms.

According to another preferred aspect of the invention the connector comprises a U-shaped portion comprising one or more breaking notches.

According to still another preferred aspect of the invention the connector is straight and comprises two tubiform elements of different outer and inner diameter, whereof a first element has an inner diameter that is slightly larger than the outer diameter of a second element. The second element is partially disposed in the lumen of the first element and comprises, at or near its end disposed in the lumen, a thin radial flange of an outer diameter corresponding to the inner diameter of the first element, the radial flange being circumferentially attached to the inner wall of the first element by gluing, welding, friction or snap connection to form a connection which will break on application of an axial force on the elements seeking to withdraw them, such as a force of from 5 to 20 N, in particular of about 10 N. To compensate for the difference in inner diameter the first section can be provided with a sleeve insert of an outer diameter corresponding to the inner diameter of the first element and an inner diameter corresponding to the inner diameter of the second element. The sleeve insert is disposed in a lumen portion of the first element not occupied by the element.

According to a further preferred aspect of the invention, the material of the connector is selected from polystyrene or polycarbonate but other medical grade polymers of similar mechanical properties may also be used.

According to the invention is furthermore disclosed an extracorporeal fluid line or tubing for connecting a transcutaneous port of the aforementioned kind implanted into a patient or a transcutaneous, peroral, pernasal, peraural, peranal or perurethral catheter to a fluid reservoir or a fluid reservoir/pump assembly, the fluid line comprising a connector of the aforementioned kind, a first flexible tube mounted at the first end of the connector and a second flexible tube mounted at the second end of the connector, a first coupling for releaseably mounting the free end of the first tube to the transcutaneous port and a second coupling for releaseably mounting the free end of the second tube to the fluid reservoir/pump assembly, so as to provide fluid communication between the fluid reservoir/pump assembly and the transcutaneous port. While Luer Lock™ couplings are preferred couplings for use in the fluid line of the invention, couplings of any suitable kind, that is, fitting to matching couplings arranged at the transcutaneous port and the reservoir/pump assembly, may be used.

The device of the invention is of a simple design facilitating its manufacture for disposable use.

According to the invention is also disclosed a method of transcutaneous, peroral, peraural, pernasal, peranal or perurethral fluid exchange in a patient, comprising providing a transdermal access port implanted into the patient or a transcutaneous, peroral, peraural, pernasal, peranal or perurethral catheter firmly attached to the patient, the port or catheter being provided with a tubing coupling means; coupling the first coupling means of the tubing of the invention to the port or catheter coupling means; exchanging fluid through the tubing and port or catheter.

Also is disclosed the use of the extracorporeal medical tubing of the invention in a method of transcutaneous, peroral, peraural, pernasal, perurethral or peranal fluid exchange with the human body.

The invention will now be explained in more detail by reference to preferred embodiments thereof illustrated in a drawing comprising a number of figures.

### SHORT DESCRIPTION OF THE FIGURES

FIG. 1 is a side view of a first embodiment of the fluid line of the invention, only the V-formed connector and short adjacent portions of soft tubing being shown;

FIG. 1a is the embodiment of FIG. 1 and in the same view, affected by a force F seeking to pull the arms of the connector apart, in a state of breaking;

FIG. 1b is the embodiment of FIGS. 1 and 1a, upon severance of the arms of the connector.

FIG. 2 is a second embodiment of the fluid line of the invention, in a state corresponding to that of FIG. 1;

FIG. 3 is a third embodiment of the fluid line of the invention, in a state corresponding to that of FIG. 1;

FIG. 4 is a fourth embodiment of the fluid line of the invention, in a state corresponding to FIG. 1;

FIG. 5 is a fifth embodiment of the fluid line of the invention, in a state corresponding to FIG. 1;

FIG. 6 is the connector of the embodiment of FIG. 1, in an axial section;

FIG. 7 is a further embodiment of the fluid connector of the invention;

FIG. 8 is a rough sketch showing the fluid line of the invention in use with a patient receiving a medication.

### DESCRIPTION OF PREFERRED EMBODIMENTS

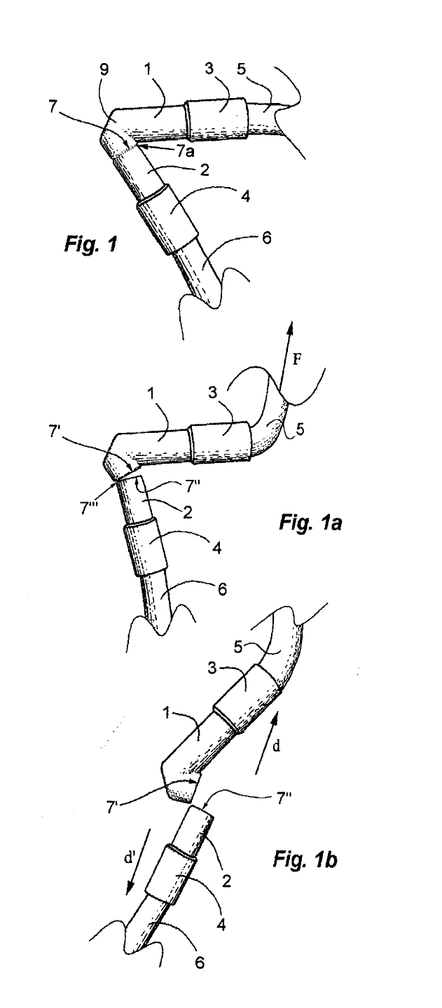

A first embodiment of the fluid line of the invention illustrated in FIG. 1 comprises a V-formed connector 10 (FIG. 6) having a lumen 11 and comprising a tubular first arm 1 and a second arm 2 with axes S and R, respectively. The angle u included by arms, i.e. their axes S and R, is about 60°. The free ends of the arms 1, 2 are provided with sleeves 3, 4, in which first 5 and second 6 flexible PVC tubes have been mounted by gluing. At their free ends (not shown) the tubes 5, 6 are provided with male/female Luer Lock™ connectors for connecting the line to a fluid reservoir/pump assembly and a transcutaneous port (not shown). The connector 10 is of a substantially non-flexible, brittle polymer material such as polystyrene or polycarbonate. The second arm 2 is provided with a predetermined breaking point in form of a circumferential notch 7, which is designed to break at a load of about 5 N acting on the joints between the sleeves 3, 4 and the flexible tubes 5, 6 so as to pull arms 1, 2 away from each other. A sudden force F of more than 5 N acting on the connector 1, 2 via the first tubing 5 results in a fracture of safety notch 7 starting at its innermost point 7a (FIG. 1a). The fractured end faces of the arms 1, 2 thus formed are designated 7′, 7″. FIG. 1b illustrates the situation just after complete severance of the arms 1, 2, the severed portions 1, 3, 5; 2, 4, 6 of the fluid line being free to move away from each other in directions d, d′, whereby the integrity of the implant in the patient is preserved. The portion 2, 4, 6 of the broken fluid line attached to the transcutaneous port can be dismounted and replaced by a substitute fluid line, which is then coupled to the existing fluid reservoir/pump assembly or a substitute assembly.

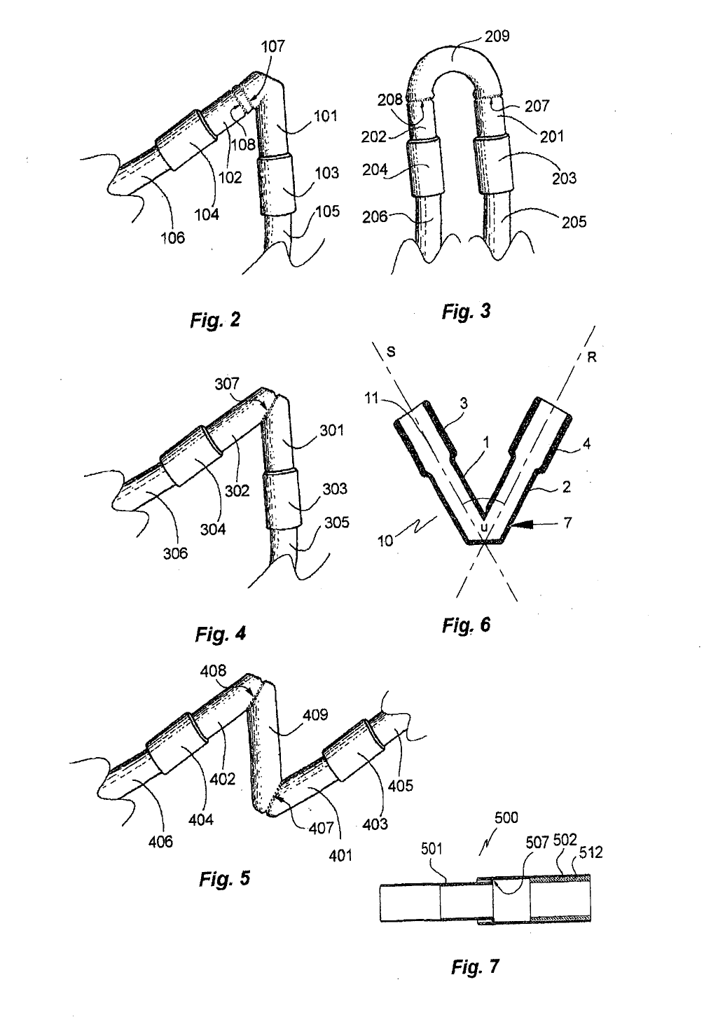

The fluid connector of the second embodiment of the fluid line of the invention shown in FIG. 2 differs from that of FIG. 1 only by having a second circumferential safety notch 108 in addition to the first circumferential safety notch 107, both radially disposed on the second tubiform arm 102 of the connector. Elements identified by reference numbers 101 and 103-105 correspond functionally to those identified by reference numbers 1 and 3-5, respectively, in the embodiment of FIG. 1.

The U-formed fluid connector of the third embodiment of the fluid line of the invention shown in FIG. 3 comprises two tubiform arms 201, 202 provided with sleeve sections 203, 204 to which soft polymer tubes 205, 206 are firmly attached. The arms 201, 202 are connected by a hemicircular tube section so as to dispose the arms 201, 202 in parallel. The joints are in form of radially disposed safety notches 207, 208.

The fluid connector of the fourth embodiment of the fluid line of the invention shown in FIG. 4 differs from that of FIG. 1 by having the safety notch 307 disposed at the joint of the first 301 arm with the second 302 arm. Reference numbers 303-306 identify elements functionally corresponding to elements 3-6 of the first embodiment of FIG. 1.

The Z-formed fluid connector of the fifth embodiment of the fluid line of the invention shown in FIG. 5 differs from that of the first embodiment shown in FIGS. 1 and 6 by comprising a central section 409 disposed between the first arm 401 and the second arm 402. The joints between the central section 409 and the first 401 and second 402 arms are in form of safety notches 407, 408. Elements 403-406 correspond functionally to elements 3-6 of the embodiment of FIGS. 1 and 6.

In contrast to the safety connectors of the preceding embodiment the safety connector 500 illustrated in FIG. 7 is straight. It comprises first and second tubiform sections 501, 502. The first section 501 is partially inserted into the lumen of the second section 502, to which it is attached by a thin circumferential flange 507 disposed at the inserted end of the first section. To compensate for the narrower lumen of the first section 501 when attaching soft flexible polymer tubes of same outer diameter to the connector, a tubiform insert 512 is arranged in a lumen portion of the second section 502 extending from the end thereof opposite to the end facing the first section 501. Application of an axial load on one section of the connector 500 seeking to displace it away from the other section will result in rupture of the circumferential flanged 507 if the other section is prevented from being displaced in the same direction and if the load is high enough to cause rupture.

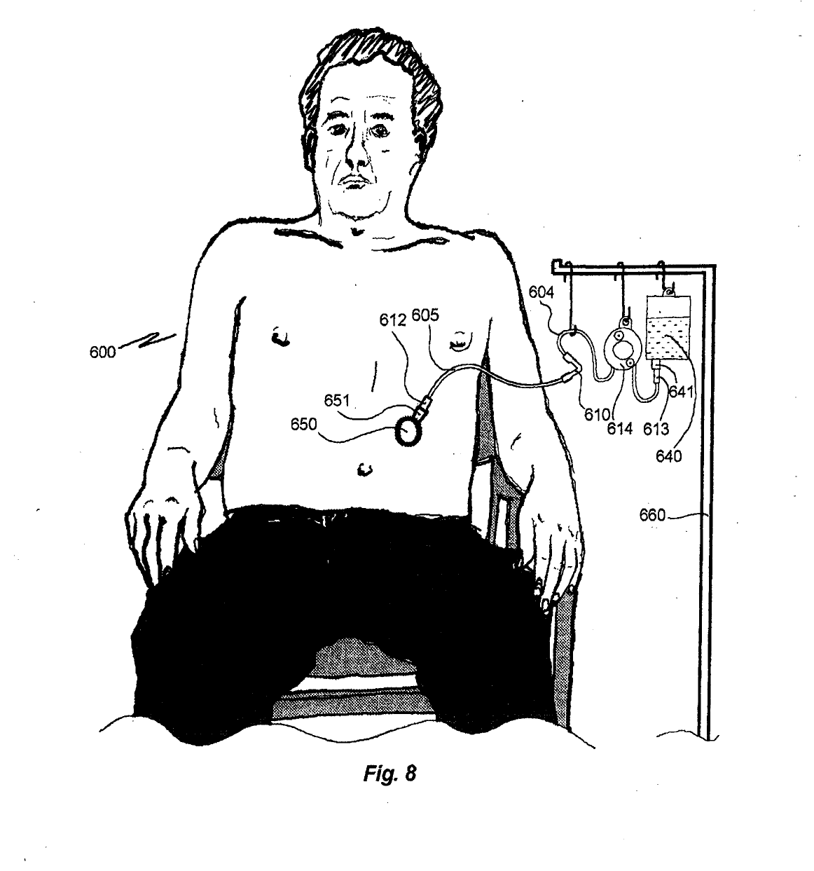

FIG. 8 illustrates the use of the fluid line of the invention with a patient 600 suffering from Parkinson's disease to whom fluid medication comprising levodopa in a translucent polymer bag 640 is administered by means of a roller pump 614 acting on a first soft polymer tube 604 coupled to the bag 640 by means of female/male Luer Lock™ coupling elements 613, 641 and firmly attached to one end of a V-formed safety connector 610 of the invention corresponding to that illustrated in FIGS. 1 and 6. To the other end of the connector 610 a second soft polymer tube 605 is firmly attached. At its other end the tube 605 is provided with a male Luer Lock™ coupling for connecting it to a corresponding female coupling arranged on the head 650 of a transcutaneous port implanted in the belly musculature of the patient. A catheter extends from the implanted portion of the port through the stomach wall into the duodenum, which is a preferred site of administration of medications like levodopa. The fluid medication bag 640, the roller pump 614 and the fluid line 604, 610, 605 of the invention are supported by a stand 660.

1. Safety fluid connector for transcutaneous, peroral, peraural, pernasal, peranal or perurethral exchange of fluid with the human body of a substantially non-flexible polymer material and having a first open end and a second open end connected by a passage for fluid, comprising a predetermined breaking point designed to fracture at a predetermined breaking force, wherein the predetermined breaking force, when applied to said ends so as to draw them apart, is from 1 N to 20 N.

2. The safety fluid connector of claim 1, wherein the predetermined breaking point comprises a failure notch.

3. The safety fluid connector of claim 2, wherein the failure notch is a notch in the external wall of a tubular section of the connector in a plane substantially perpendicular to the longitudinal axis of said section.

4. The connector of claim 1, of V or U shape or comprising a V- or U-shaped section.

5. The safety fluid connector of claim 4 of V shape or comprising a V-shaped section, wherein the arms of the V include an angle of from 15° to 75°.

6. The safety fluid connector of claim 1 of straight configuration, comprising first and second tubiform elements of different outer and inner diameter, wherein the first element has an inner diameter that is slightly larger than the outer diameter of the second element and the second element is partially disposed in the lumen of the first element and comprises, at or near its end disposed in the lumen, a thin radial flange of an outer diameter corresponding to the inner diameter of the first element, the radial flange being circumferentially attached to the inner wall of the first element to form said predetermined breaking point.

7. The safety fluid connector of claim 1 of polycarbonate or polystyrene or a material of similar mechanical properties.

8. Extracorporeal medical tubing comprising the safety fluid connector of any of claim 1 non-releaseably connected at its first end to one end of a first flexible tube comprising a first coupling means, such as of Luer Lock® type, at its other end.

9. The extracorporeal medical tubing of claim 8, non-releaseably connected at its second end to one end of a second flexible tube comprising a second coupling means, such as of Luer Lock® type, at its other end.

10. A method of transcutaneous, peroral, peraural, pernasal, peranal or perurethral fluid exchange in a patient, comprising:
providing a transdermal access port implanted into the patient or a transcutaneous, peroral, peraural, pernasal, peranal or perurethral catheter firmly attached to the patient, the port or catheter being provided with a tubing coupling means;
providing the extracorporeal tubing of claim 9;
coupling the first coupling means of the tubing to the port or catheter coupling means; and
exchanging fluid through the tubing and port or catheter.

11. Use of the extracorporeal medical tubing of claim 8 in transcutaneous, peroral, peraural, pernasal, peranal or perurethral fluid exchange with the human body.

12. The safety fluid connector of claim 1, wherein the predetermined breaking force, when applied to said ends so as to draw them apart, is from 5 N to 20 N or 5 to 15 N.

13. The safety fluid connector of claim 1, wherein the predetermined breaking force, when applied to said ends so as to draw them apart, is about 10 N.

14. The safety fluid connector of claim 4, wherein the arms of the V include an angle from 5° to 60°.

15. The safety fluid connector of claim 4, wherein the arms of the V include an angle from 10° to 45°.
